(19) United States
(12) Patent Application Publication
(10) Pub. No.: US 2023/0181096 A1
(43) Pub. Date: Jun. 15, 2023

(54) SYSTEMS AND METHODS FOR POWERING AUTONOMOUS SWEAT SENSOR

(71) Applicant: California Institute of Technology, Pasadena, CA (US)

(72) Inventors: Wei Gao, Pasadena, CA (US); Jihong Min, Pasadena, CA (US)

(21) Appl. No.: 18/077,846

(22) Filed: Dec. 8, 2022

Related U.S. Application Data
(60) Provisional application No. 63/287,894, filed on Dec. 9, 2021.

(51) Int. Cl. A61B 5/00; G06F 1/16; H02S 40/30; H02N 1/04; H05K 1/18

(52) U.S. Cl. CPC A61B 5/4266; G06F 1/163; H02S 40/30; H02N 1/04; H05K 1/189

(57) ABSTRACT

Systems and methods for a self-powered wireless wearable sensor system include a photovoltaic (PV) panel array, used as a power source for a wearable sensor. The PV panel array may be attached to an area of the human body exposed to a light source. Exposure to a light source may generate an electric field and power a wearable device sufficiently to support data transmission and continuous monitoring. An integrated self-powered wireless wearable sensor system may include a microfluidic sweat sensor patch that may be connected to lower-power wireless sensor circuitry for regulating power efficiently and may be powered by the PV panel array.

### RELATED APPLICATION

This application claims the benefit of U.S. Provisional Application No. 63/287,894 filed on Dec. 9, 2021, the contents of which are incorporated herein by reference in their entirety.

### TECHNICAL FIELD

The present disclosure relates generally to systems and methods for powering a wearable device. In particular, some implementations may relate to methods for powering a wearable device using a photovoltaic power source, such that the device may function autonomously.

### BACKGROUND

Wearable bioelectronic technology offers many advantages for personalized health monitoring. Wearable devices are non-invasive and present less user error than other monitoring methods. Additionally, wearable devices offer the potential to monitor health status over time as opposed to collecting a sample that reflects health status at only a snapshot in time. This type of real-time monitoring offers more accurate and individualized diagnosis, treatment, and prevention for health conditions. Specifically, wearable devices can measure pulse, respiration rate, temperature, and other health status indicators.

Sweat sensors are one type of wearable bioelectronic sensors that are particularly desirable because sweat contains many key biomarkers including electrolytes, metabolites, amino acids, hormones, and drug levels. However, existing sweat sensors face several key problems. These sensors often require a large sample of sweat to provide accurate analysis of biomarkers. This requires a larger and more powerful device that may not be suitable as a wearable device. Additionally, existing sweat sensors have high-power demands. Therefore, monitoring, and especially continuous monitoring, presents a challenge. Existing models are limited in the amount of time they can operate continuously due to their power consumption and limits on power storage. Existing models present additional challenges, including that they require complex fabrication and are difficult to reproduce in large quantities in an affordable way. They are also fragile and not suitable as wearable devices for long periods. They also suffer from low-power density. Because wearable devices must be small and light-weight practically, high-power density is an important characteristic of an efficient and effective wearable device.

Due to the high-power demands required, currently existing wearable health monitoring systems are typically powered by batteries. Many types of batteries add weight and bulk to the device. Some also pose the risk of burns. Even lightweight batteries have significant drawbacks, such as needing to be charged and replaced frequently.

Additionally, typical photovoltaic (PV) technologies, such as silicon, may not be effective in a wearable device. For example, typical PV technologies may suffer from several defects including fragility, bulkiness, rigidity, and inadequate power supply under indoor lighting conditions. These defects may render typical PV technologies unsuitable for powering wearable devices. Other typical PV technologies, such as III-V light harvesters, may be too complex and expensive to fabricate for a wearable device application.

Because of the lack of effective continuous monitoring strategies and high-power demands, currently existing wearable health monitoring systems are unable to measure key biomarkers over extended periods of time. An effective wearable system would be highly desirable as alternatives, such as blood testing, are invasive, expensive, and offer limited information over time.

### SUMMARY

Systems and methods are described herein for a self-powered wireless biosensor system. Such autonomous methods offer advantages over batteries. Autonomous powering methods include powering from human motion, powering from biofluids, and powering from light sources whether solar or artificial. However, generally available autonomous powering methods may not be sufficient to meet the efficiency and power demands for powering a wearable biosensor device.

One type of autonomous power method may use photovoltaic (PV) panels. PV panels include small PV cells fabricated using semiconducting material, such as silicone. When exposed to light, PV cells generate an electric field, converting light energy into electric energy. Many PV panels capture light energy from the sun; however, indoor light sources may also serve as a power source. Therefore, operation of devices powered with PV panels may offer advantages over other energy sources such as batteries, which might run out. However, PV panels may suffer from low-power density, inefficient power management, and a lack of power continuity and longevity. For instance, a relatively large panel or configuration of panels may be needed to capture sufficient energy to power even a small device. Such a large assembly may be too large to work well with a wearable device. Additionally, external conditions, such as the sun being obscured or it being night, may reduce or cut off the supply of light energy, leading to intermittent powering and/or energy storage concerns that would not be suitable for a wearable device performing continuous monitoring. Accordingly, conventional photovoltaic panel based power sources have been unable to meet the power demands of a continuously monitoring wearable biosensor.

Embodiments of the present disclosure provide a photovoltaic power system for a wearable device. A PV power system may include PV panels, supporting circuitry, and a wearable sensor. The PV power system may be a lightweight array limited to a threshold surface area for each application to the human body. The PV panels may include high efficiency PV panels such that the panels may effectively power the system using indoor and/or artificial light alone. The supporting circuitry may efficiently manage and store the power generated to supply a stable voltage over a period of up to several weeks. Additionally, the wearable sensor may have lower powering needs than predecessor sensors. For instance, it may require a smaller sample which may be induced and processed using less energy. All of these features contribute to allowing the device to be powered using an autonomous energy source, such as the sun or artificial light.

A photovoltaic power system for a wearable device must be carefully designed to ensure sufficient power is achieved to power the wearable device and/or to enable the wearable device to perform continuous monitoring over a period of time. Sufficient power may also enable the wearable device to transmit data to a user interface or another source where the data can be viewed and analyzed. For example, a mobile application may present a user interface on a mobile device. Data may be transmitted to the mobile device via Bluetooth.

To ensure a photovoltaic power system for a wearable device is compatible with wearable devices and/or related circuitry, and that the power system can withstand long term use without compromising its ability to supply needed power, supporting circuitry for the photovoltaic panels may be fabricated using printed circuit board (PCB) technology. The supporting circuitry may include an energy harvesting power management integrated circuit (PMIC). The PMIC may efficiently boost, convert, and manage power output from the photovoltaic cells. The supporting circuitry may also include a compact programmable BLE module. The BLE module may integrate a microcontroller (MCU) and a BLE radio. The supporting circuitry may also include a high compliance voltage current source for iontophoresis. The supporting circuitry may also include an electrochemical analog front-end (AFE) chip. The AFE chip may integrate various configurable blocks necessary for electrochemical detection.

The wearable sensor patch of the system may be a microfluidic sweat sensor patch. Sweat may contain many indications of health including ion concentrations, amino acid levels, hormone levels, vitamin and mineral levels, presence of drugs, and other indicators of health. A microfluidic sweat sensor may collect a sweat sample from a sweat gland in a reservoir. The sample may be periodically refreshed. The microfluidic sweat sensor patch may allow for continuous monitoring of health indicators over a period of time. As the sweat samples refresh, the new samples may reveals changes or trends in the body. A sweat sample may operate using a small amount of sweat and may not have significant power needs compared with other types of biosensors. A sweat sensor may also be non-invasive so that a human subject may be comfortable wearing a sweat sensor patch over a period of time. A sweat sensor patch may also be fabricated inexpensively and may be disposable such that a human subject may periodically replace a sweat sample patch as needed.

A self-powered wearable system may also include a user interface. A user interface may be available on a mobile device, for instance via an application. A user may access data collected from a wearable biosensor via the user interface. Data collected from a wearable biosensor may be transmitted such that it can be accessed via the user interface using a wireless method, such as Bluetooth. The photovoltaic panel may supply sufficient power for a Bluetooth transmission or another type of wireless transmission of data.

The photovoltaic panel powered system may be configured to power and support different types of wearable sensor patches. For instance, identical disposable sweat sensor patches may be replaced without compromising the system effectiveness. Alternatively, sweat sensor patches having different functions, e.g., one that measures hormone levels and one that measures amino acid levels, may both be compatible with the system. Alternatively, a different type of biosensor altogether, such as a body temperature sensor, may be connected to the system.

A PV panel powering system may be combined with another type of powering system to achieve even greater energy stores for the device and to compensate for times when one or more powering sources is not available. One type of other powering system may be an FTENG. An FTENG converts mechanical energy into electrical energy by inducing charge when movement occurs. For example, the FTENG may include one or more interdigital stator panels and one or more grating patterned slider panels. The slider may move from a first position relative to the stator to a second position relative to the stator, inducing a charge when a human subject wearing the wearable device engages in certain types of cardiovascular exercise. This enables the system to be powered even when other power sources, such as battery power, conventional electrical power provided via an outlet, or the sun, are not available.

Another type of powering system may be a powering system that harnesses heat energy and converts heat energy into electrical energy. For example, heat energy from an external source, such as the sun, or body heat from a human subject may produce sufficient energy to power the devices alone and/or in conjunction with one or more other types of powering systems. For example, a thermionic generator may use a temperature difference between a hot and cold metallic plate to create electricity. Careful selection of metallic materials combined with heat energy from, for example, the human body, may be sufficient to create the kind of temperature differential needed for such a device to generate electric energy.

A method for powering a wearable device may comprise wearing the wearable device. The wearable device may include a PV panels. The PV panels may arranged in a way that is suitable for application to the human body. For example, a small PV panel array may be applied to a human subject's arm or wrist. The surface area of a small PV panel may be similar to that of a smart watch and may be worn by a human subject using a band in a similar configuration. Alternatively, or additionally, a larger PV panel may be worn on a human subject's torso. For example, a larger PV panel may be applied to a human subject's back area using a strap, medical adhesive, or another mechanism. Alternatively, a PV panel may be applied to any other human body area exposed to a light source.

A method for powering a wearable device may also comprise exposing a wearable device to a light source. The wearable device may be applied to any area on the human body that is exposed to a light source. For example, the wearable device may be applied to a human arm or a human torso. Exposing PV panels to a light source will create an electrical field which may be sufficient to power a wearable device. A supporting circuitry may manage and store the generated energy to power a wearable device over a long period of time. For example, captured and stored energy may power a wearable device for a period of up to several weeks. A wearable device may include a sensor which captures health data continuously over a period of time. The health data may be transmitted to a mobile device having a user interface. The health data may be transmitted via Bluetooth. The method may also include a further step of accessing sample data collected by a wearable device using a user interface.

Other features and aspects of the disclosure will become apparent from the following detailed description, taken in conjunction with the accompanying drawings, which illustrate, by way of example, the features in accordance with various embodiments. The summary is not intended to limit the scope of the invention, which is defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a diagram showing an example of a self-powered wearable biosensor system.

FIG. 2A is a diagram showing an example of a stator for use in a self-powered wearable device.

FIG. 2B is a diagram showing an example of a slider for use in a self-powered wearable device.

FIG. 3A is a diagram showing an example of a stator and slider for use in a self-powered device showing the slider occupying a first position relative to the stator.

FIG. 3B is a diagram showing an example of a stator and slider for use in a self-powered device showing the slider as it moves from a first position to a second position.

FIG. 3C is a diagram showing an example of a stator and slider for use in a self-powered device showing the slider occupying a second position relative to the stator.

FIG. 4A is a diagram showing an example of a stator and slider for use in a self-powered device showing the slider occupying a first position relative to the stator.

FIG. 4B is a diagram showing an example of a stator and slider for use in a self-powered device showing the slider as it moves from a first position to a second position.

FIG. 4C is a diagram showing an example of a stator and slider for use in a self-powered device showing the slider occupying a second position relative to the stator.

FIG. 5A is a diagram of an example of a stator and a slider in a one-panel configuration.

FIG. 5B is a diagram of an example of a stator and a slider in a three-panel configuration.

FIG. 5C is a diagram of an example of a stator and a slider in a six-panel configuration.

FIG. 6A is a diagram showing an example of a microfluidic sweat sensor patch.

FIG. 6B is a diagram showing an example of a microfluidic sweat sensor patch applied to a human body.

FIG. 7 is a diagram showing an example of a low-power wireless sensor circuitry for managing power supplied by an FTENG to power a biosensor device.

FIG. 8 is a diagram showing an example of a system including circuitry for managing power supplied by both photovoltaic panel(s) and a TENG device to power a biosensor device.

FIG. 9 is a diagram showing an example supporting circuitry for managing power supplied by photovoltaic panel(s) to power a biosensor device.

FIG. 10A is a diagram showing an example of a self-powered wearable biosensor system applied to a human body.

FIG. 10B is a diagram showing an example of a slider panel worn by a human being.

FIG. 11 is a diagram showing an example of a self-powered wearable biosensor system applied to a human body.

FIG. 12 is a flow diagram showing an example of a method for self-powering a wearable biosensor system.

FIG. 13 is a flow diagram showing an example of a method for self-powering a wearable biosensor system.

FIG. 14 is a diagram showing an example of the circuitry of an electrochemical analog front-end (AFE) chip.

FIG. 15 is a diagram showing an example of a disposable microfluidic sweat patch for sweat induction and sampling.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION

Wearable devices may offer highly desirable, non-invasive, and continuous monitoring of key health indicators. However, these devices are difficult to design since health monitoring and especially continuous health monitoring can have high-energy demands. One type of desirable wearable is a sweat sensor. A carefully and efficiently designed system may enable autonomous powering of a sweat sensor. Several types of autonomous powering are available including powering by human motion.

The embodiments described herein relate to a battery-free, fully self-powered wearable bioelectric medical monitoring system. The system may include an autonomous power source, circuitry, and a sensor patch. In an embodiment, the sensor patch may be a microfluidic sweat sensor. In an embodiment, the autonomous power source may be a photovoltaic panel. This is an example of the type of integrated system that may solve industry issues regarding self-powering of a wearable biosensor system. Other types of systems are also possible and this example is not intended to be limiting.

### Photovoltaic Panels

A PV panel for use in powering a wearable biosensor may include many small PV cells fabricated using a semiconducting material, such as silicone. When exposed to light, PV cells may generate an electric field, converting light energy into electric energy. The PV panels may capture light energy from the sun. Alternatively, the PV panels may capture light from an artificial source, such as indoor lighting. High efficiency PV panels may generate sufficient amounts of energy to power a wearable biosensor using artificial light alone and/or minimal sun exposure. Additionally, an array of high efficiency PV panels having a small surface area may generate sufficient energy to power a wearable biosensor.

Materials for photovoltaic panels may be carefully selected to ensure the photovoltaic panels will be suitable for use as part of a wearable biosensor system. Specifically, the photovoltaic panels should be flexible so they can either be attached to the human body and/or integrated into a wearable device that a human being may wear during exercise or other activities. Additionally, the photovoltaic panels may be able to achieve a relatively high-power density to power a wearable biosensor system, so they should be both efficient and lightweight. Certain materials may offer these desirable properties. For example, thin-film solar cell modules may be lightweight, offer mechanical flexibility, and be moldable. A thin-film solar cell module may include a flexible substrate that may include different materials. In some embodiments, such materials may be deposited via a printer.

Flexible substrates may include thin metals, ceramics, such as ultra-thin glass, and plastics. The following example materials may be used in addition to other carefully selected materials: polycarbonate, zirconia, polyethylene naphthalate, polyethylene terephthalate, titanium, polyimide, stainless steel, aluminum, and molybdenum. Active semiconductor materials may also be lightweight, flexible, and efficient. For example, active semiconductor materials may include hydrogenated amorphous silicon, Cu(In,Ga)Se (CIGS), organic semiconductors, and perovskite active materials. Methylammonium chloride (MACI) may also be added to the perovskite layer. The addition of MACI may increase grain size and reduce defect, improving the efficiency and stability of the solar cell. In one embodiment, an inorganic-organic semiconductor including metal halide perovskite may be used and may offer desired flexibility. A flexible perovskite solar cell may be conformable to the skin, sufficiently durable for wearing on the human body during exercise, including through exposure to moisture such as sweat, and may yield a sufficient power density to power the device. A perovskite cell, as described above, may function well under both natural and artificial light. In an embodiment, a perovskite cell may achieve superior functioning under indoor illumination. A perovskite cell may also be packaged to function under water without lead leakage.

Perovskite solar cells may offer many favorable properties, including long charge carrier diffusion length, high absorption coefficient, solution processability, small exciton binding energy, high structural defect tolerance, tunable bandgap, and high photo luminescence quantum yield. These properties may make perovskite solar cells a desirable choice for powering a wearable device, making the device a self-powered wearable device. For example, perovskite's defect tolerance may lead to high shunt resistance (HSR) that may allow for operation of a solar-powered system even under low light conditions. Specifically, high defect tolerance may result in increased fill factor (FF) and reduced open circuit voltage (VOC) losses at low light conditions. This, in combination with matching of perovskite solar cells' spectral response to common indoor lighting emission spectrum, may yield higher power conversion efficiency (PCE) under indoor illumination.

In an embodiment, a photovoltaic powering system may also include anti-reflective coatings. An anti-reflective coating may include a thin dielectric material with a carefully selected thickness. Light waves reflected from the coating layer may then be out of phase with light waves reflected from the semiconductor layer. In this way, the coating may produce a destructive interference resulting in a net zero of reflected light energy. An anti-reflective coating may increase the efficiency of a photovoltaic system by preventing and/or reducing energy loss. Example dielectric coating materials may include silico nitride and titanium dioxide, among other carefully selected dielectric coating materials. In another embodiment, a photovoltaic system may include protective coatings. For example, protective coatings may include materials that repel water and dust, which may minimize damage to the cells. Other coatings may prevent fogging and/or obscuration of the photovoltaic panels. In embodiments, anti-reflective and protective coatings may be combined.

One embodiment may include a photovoltaic powering system that can use heat as a power source. Traditional photovoltaic systems may operate by absorbing light at the visible spectrum. However, another type of photovoltaic system may instead use light emitted in the thermal infrared spectrum as a power source. At this spectrum, standard photovoltaic systems become inefficient. However, the combination of using infrared light and non-traditional methods of generating current may retain sufficient energy. For example, one method of generating current may be photon-assisted tunneling. This may involve confining current in a thin silicon dioxide tunnel, which may result in collecting electrons in wells, which may offer the potential for increased voltages.

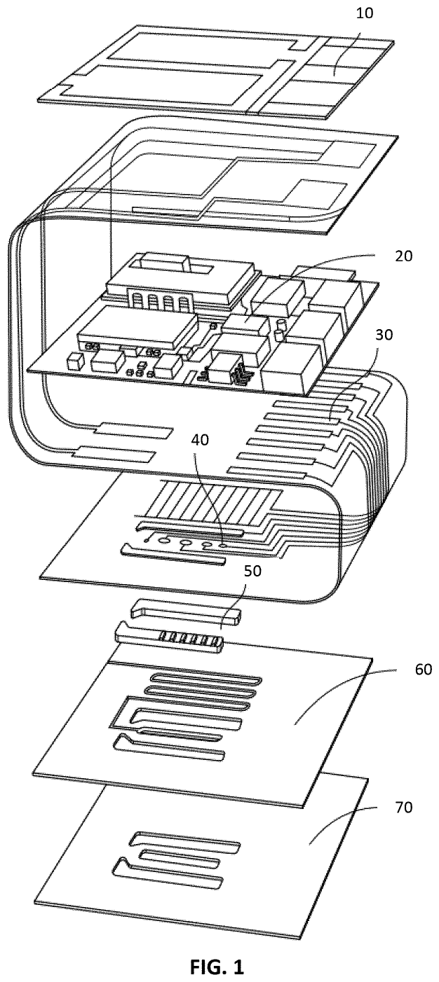

Referring now to FIG. 1, which is an example of a wearable biosensor system including a perovskite solar cell 10. The perovskite solar 10 cell may have an inverted planar heterojunction architecture, which may include a PET substrate, an ITO transparent conductive oxide layer, a PEDOT:PSS hole transport layer, an organic-inorganic hybrid perovskite absorber layer, a PTCDI electron transport layer, a chromium oxide interlayer followed by a gold contact layer, and a polyurethan encapsulation layer. The perovskite solar cell 10 may be connected to a flexible printed circuit board (PCB) 20. The flexible PCB 20 may be connected to a sensory array 40 via patterned contacts 30. The sensory array may include carbachol 50 to stimulate the production of sweat for collection. The wearable system may also include a microfluidic layer 60 attached to the sensory array to collect sweat samples. The wearable system may also include an adhesive layer 70 connected to the microfluidic layer 60 and attached to the human body.

A flexible perovskite solar cell (FPSC), as shown in FIG. 1, for example, may be designed to have a high-power density and PCE for energy harvesting under diverse lighting conditions, stable and prolonged performance with reliable encapsulation against extended sweat exposure, and flexibility to endure the mechanical stress common for on-body performance. The FPSC may utilize a p-i-n architecture and may be comprised of flexible polyethylene terephthalate (PET) substrate coated with an indium tin oxide (ITO) transparent conductive layer, a poly (3,4-ethylenedioxythiophene) polystyrene sulfonate (PEDOT:PSS) hole transport layer, quasi-2D perovskite photoactive layer, [6,6]—phenyl-C61-butyric acid methyl ester (PCBM) electron transport layer, TiOx interlayer, followed by a Cr/Au contact, and a PVC/PCTFE encapsulation film in some embodiments. In some embodiments, a Cr/Au busbar may also be introduced between ITO and PEDOT:PSS to enable more efficient charge collection and lower series resistance (Rs). In some embodiments, a solar cell may also include a perovskite absorber layer. The perovskite absorber layer may have a thickness of about 450 nm and an empirical formula of (MBA)2(Cs0.12MA0.88)6Pb7(IxCl1−x)22. In some embodiments, a large organic spacer molecule, for example, α-methylbenzylamine (MBA), may facilitate the formation of a quasi-2D perovskite structure with increased grain size and improved defect passivation, which may result in an enhanced device performance and stability. In some embodiments, under about 1 sun (AM1.5) illumination quasi-2D perovskite solar cells with an active area of about 0.18 cm² may achieve PCE of above about 18%. The PCE may remain as high as about 16.76% when the active area is scaled up to about 1 cm². In order to assemble FPSC modules, two cells with about 1 cm² active area may be connected in series that then deliver up to about 28.02 mW with a PCE of about 14.01%.

In some embodiments, an FPSC, as described above and shown in FIG. 1, for example, may enable powering via indoor lighting sources. Common indoor lighting sources, such as, for example, LED light bulbs, may have a narrower spectrum and lower photon flux density when compared to sunlight. The external quantum efficiency (EQE) of a quasi-2D FPSC may closely match the emission spectrum of an ordinary off-the shelf LED light bulb, which may allow for an increase in efficiency due to reduced sub-bandgap and relaxation losses. While low light intensity can be detrimental to solar cell performance due to the presence of interfacial trap states and grain boundaries, the incorporation of α-methylbenzylamine (MBA) as a large organic spacer molecule may allow for superior passivation of these defects. As a result, a quasi-2D FPSC, for a large area of about 1 cm² devices, may reach a PCE of about 42.7% and of about 42.4% for an about 2 cm² sized modules under about 610 1× illuminance using a 2700 K LED light bulb. Furthermore, the power output of the quasi-2D FPSC module may yield a reliable performance over a broad range of indoor illuminance from very bright (approximately 10000 1×) sources, common for special environments like surgery rooms, down to dimly lit conditions of hallways and elevators (approximately 20 1×).

### FTENG

A freestanding triboelectric nanogenerator (FTENG) for use in powering a wearable biosensor system may include a stator and a slider. The FTENG may use tribo-pairs to obtain a strong electrification effect when the slider slides across the surface of the stator. These pairs may be copper and polytetrafluoroethylene (PTFE). The stator and the slider may be coated with such materials or with other materials demonstrating desirable triboelectric properties. The FTENG may be fabricated with flexible printed circuit board (FPCB) technology. FPCB fabrication ensures the FTENG should not be compromised even when applied to the human body as part of a wearable system. It also may allow the FTENG to be compatible with other system components, including circuitry and one or more wearable biosensor devices.

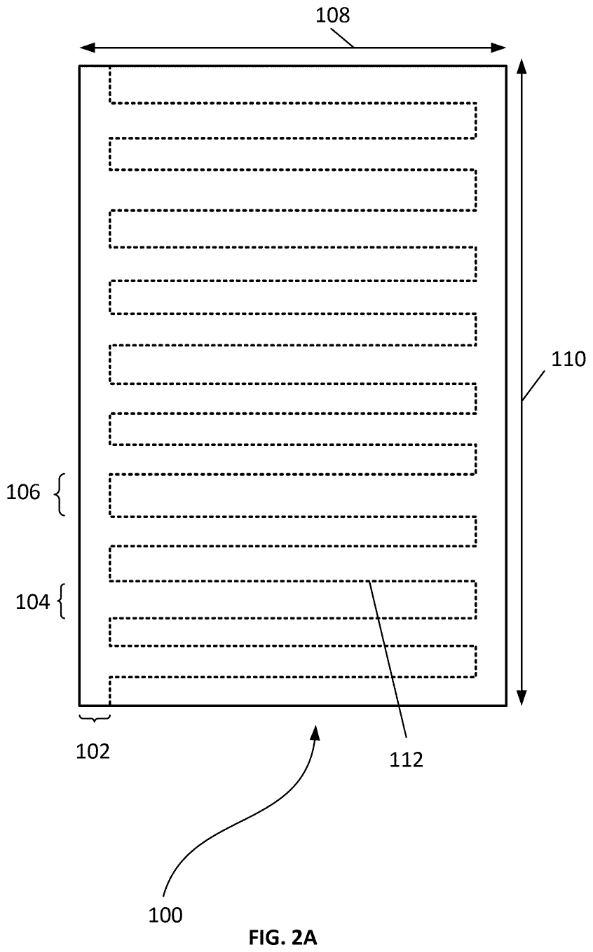

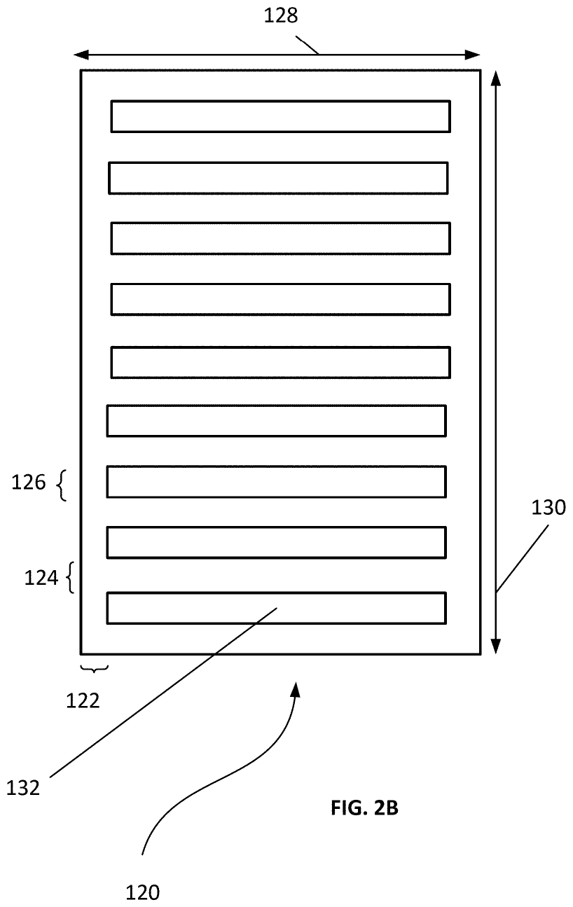

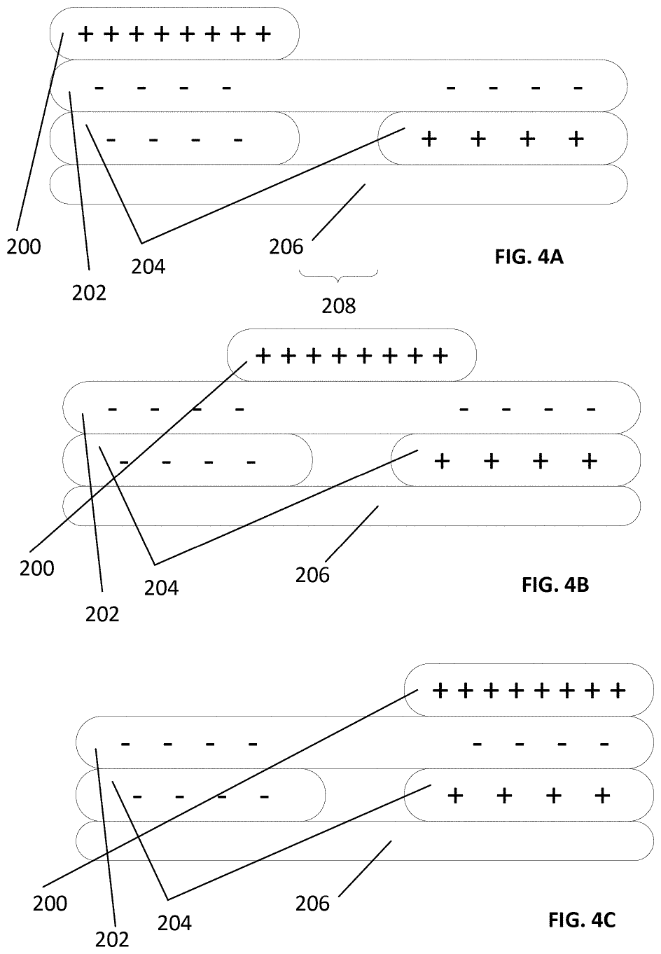

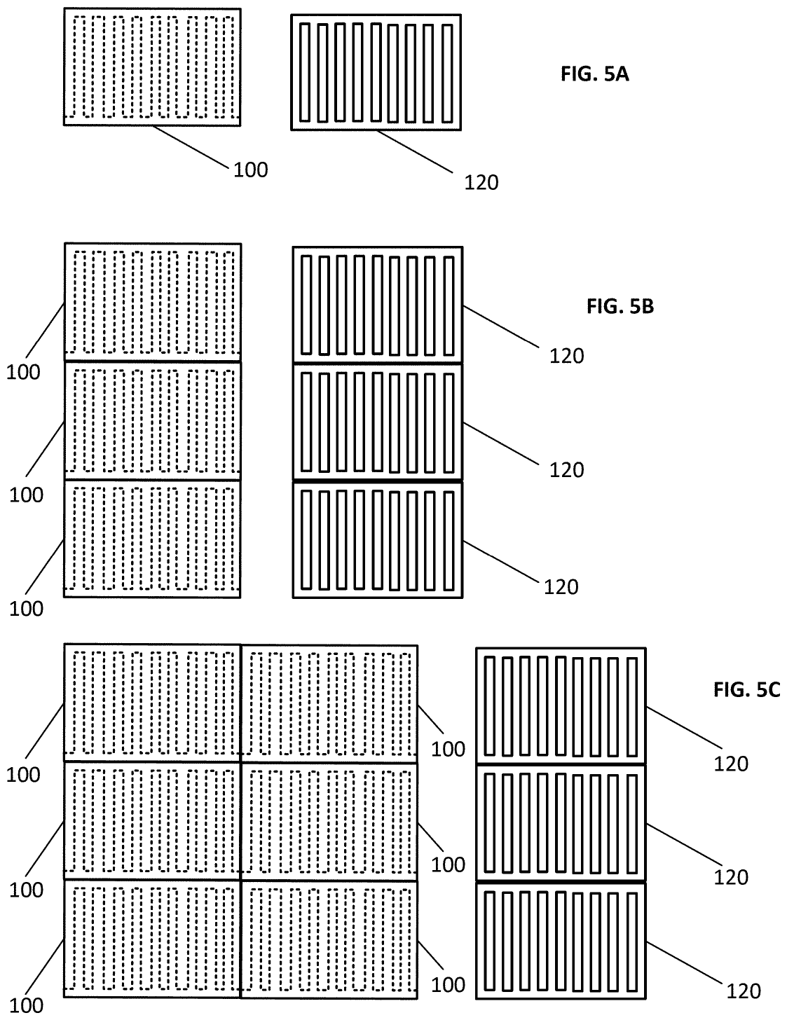

FIG. 2A depicts a diagram showing an example of a stator 100. The stator 100 may include electrodes 112 forming an interdigital structure. The stator 100 may also include an inter-electrode (for example 208 in FIG. 4) distance that may be optimized for efficient power generation. The stator 100 may include other dimensional parameters, such as height 102, gap 104, offset 106, width 108, and length 110.

FIG. 2B depicts a diagram showing an example of a slider 120. The slider 120 may include a grating pattern including several open grates 132. The slider 120 may also include dimensional parameters such as height 122, grate width 126, distance between grates 124, width 128, and length 130. When the slider 120 is placed on top of the stator 100, the grating pattern of the slider 120 and the interdigital structure of the stator 100 may be periodically complimentary.

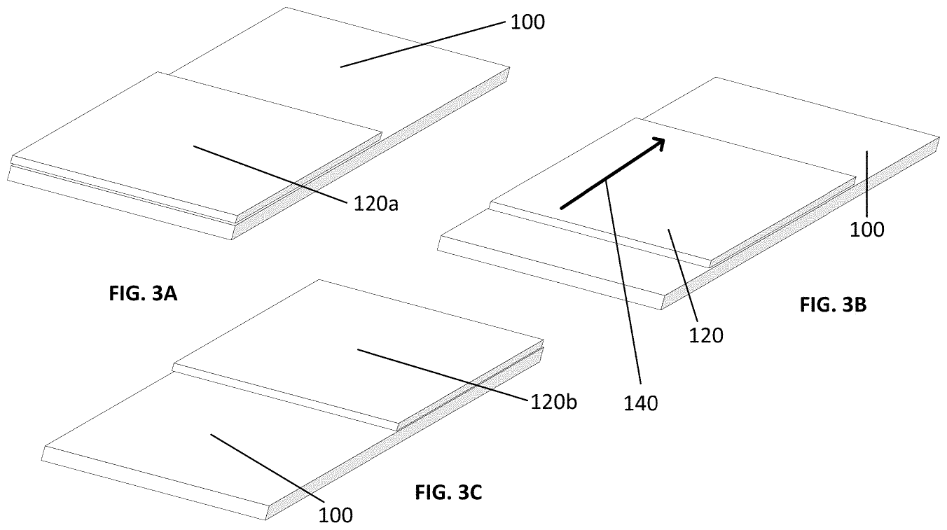

As shown in FIGS. 3A, 3B, and 3C, the slider 120 may be slidably coupled to the stator 100, enabling movement between a first position of the slider 120a and second position of the slider 120b relative to the stator 100. In the first position of the slider 120a, shown in FIG. 3A, the grating pattern (132 of FIG. 2B) of the slider 120 may fully or partially overlap with a stator electrode (112 of FIG. 2A). The system may be in electrostatic equilibrium and have no charge flowing through the electrode 112. As shown in FIG. 3B, the slider 120 may slide relative to the stator 100 in a sliding direction 140. As the slider 120 slides across the stator 100, charge may flow between the stator electrode(s) 112 until the slider 120 arrives in the second position of the slider 120b relative to the stator 100. FIG. 3C shows the slider 120 occupying the second position of the slider 120b relative to the stator 100. In this position, the grating pattern 132 of the slider 120 may fully or partially overlap with the second electrode 112 of the stator 100. The second electrode 112 of the stator 100 may have reversed polarity.

FIG. 4 shows examples of the slider and stator in similar positions as to those shown in FIG. 3, but from a front perspective (FIG. 4A corresponds to FIG. 3A's position; FIG. 4B corresponds to FIG. 3B's position; FIG. 4C corresponds to FIG. 3C's position). FIG. 4 also shows the triboelectric material coating the stator 100 and slider 120. For example, FIGS. 4A, 4B, and 4C shows a copper coating 200 on the slider. The copper coating 200 may have a strong positive charge. FIGS. 4A, 4B, and 4C also show a PTFE coating 202 on the stator 100. The PTFE coating may be less positively charged than the copper coating. FIGS. 4A, 4B, and 4C also show an electroless nickel/immersion gold (ENIG) surface finish 204 on the electrode 112 area of the stator 120 and a polyimide base 206. The polyimide material on the polyimide base 206 may be durable, which may allow the FTENG to be integrated into a wearable system appropriate for continuous monitoring of the human body over periods of time without degradation of the components.

Contact electrification may occur when certain materials become electrically charged after they have contact with a different material and then are separated from that other material. This may be referred to as the triboelectric effect. Different materials have different triboelectric properties that are affected by the triboelectric effect. Copper, for instance, may be more triboelectrically positive than other materials, such as polytetrafluoroethylene (PTFE). Therefore, when copper comes into contact with PTFE, electrons may be repelled from the copper and may accumulate on the PTFE. In some embodiments, PTFE may be used because it may resist scratching and degrading over time.

In embodiments, the FTENG may operate at varying frequencies. These frequencies may correspond to maximum currents. For example, such frequencies and currents may be included in Table 1:

| Frequency (Hz) | 0.5 | 1.25 | 3.3 |
|---|---|---|---|
| Current (μA) | 8.39 | 19.11 | 42.25 |

With a load resistance of approximately 4.7 MΩ and actuation frequency of approximately 1.5 Hz, the FTENG may achieve a power output of approximately 0.94 mW.

FTENGs having 1-panel, 3-panel, and 6-panel configurations may be used to power wearable devices. In other embodiments, different panel configurations may exist. FIGS. 5A, 5B, and 5C show example FTENG configurations. FIG. 5A depicts an example 1-panel configuration. FIG. 5B depicts an example 3-panel configuration. And FIG. 5C depicts an example 6-panel configuration. In various embodiments, the panels (for example the stator 100 and slider 120) may be disposed adjacent to one another. Other placements and configurations of panels may exist. For example, in embodiments, a 3-panel FTENG actuated at a working frequency of approximately 1.5 Hz may repeatedly charge an approximately 47 g capacitor over approximately a two-hour period from approximately 0 to approximately 2 V. A plurality of FTENGs may be connected in parallel or series to achieve increased power output.

### Sweat Sensor Patch

A sweat sensor patch may include a biosensor array for sweat analyte or metabolite analysis. The analysis may be based on ion-selective electrodes. The sweat sensor patch may include laser engraved microfluidic channels. The electrodes may have different coatings including polyvinyl butyral (PVB) that may maintain a steady potential to measure electrolytes in the sweat. Other coatings may exist. The sweat sensor may also measure pH and salt concentration. The sensor may measure other ion concentrations. The sensor may be configured to make other health measurements including amino acid levels, hormone levels, and drug levels.

The sweat sensor patch may be fabricated with laser patterned microfluidic lasers and may be easily reproduced. The sensor may also be flexible, which may allow for attachment to the human body without comprising the structure of the sensor. The sensor patch may be attached to the human body with medical adhesive or via other adhering methods. The sweat sensor may take continuous biologic measurements over a period of time. In some embodiments, the sweat sensor may detect changes in the human body and reflect updated measurements within a period, for example minutes, of the change.

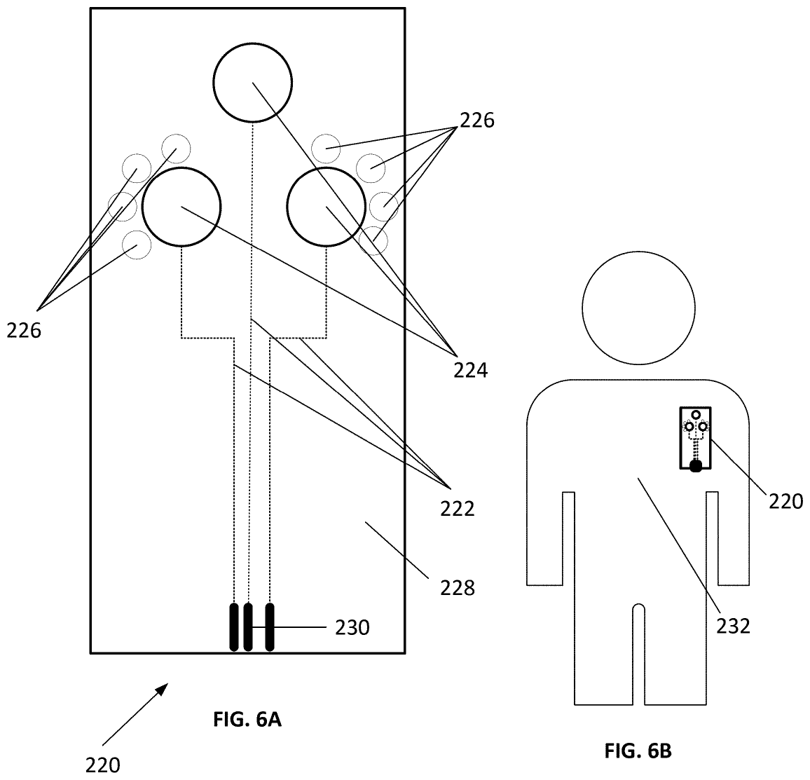

FIG. 6A shows an example of a sweat sensor patch 220. The sweat sensor patch may include laser engraved microfluidic channels 222. It may also include reservoirs 224 to collect and analyze sweat samples. The reservoirs 224 may have outlets 226 placed near several neighboring sweat glands that may be induced to produce samples. The sweat sensor patch 220 may be applied to human skin using a medical tape layer 228 or similar adhesive. For example, laser patterned microfluidic layers containing laser engraved microfluidic channels 222 may be attached to a polyethylene terephthalate (PET) sensor substrate in a layered structure so that the microfluidic chip layer lies between two layers of medical tape 228. This configuration may prevent leakage and secure the sensor to the human body. The sweat sensor patch 220 also may include a circuitry connection point 230 for integration into a self-powered sweat sensor system.

FIG. 6B shows an example of a sweat sensor patch 220 applied to a human body 232. The sweat sensor may be applied to several areas on the human body 232. In embodiments, the sweat sensor may be applied to the human torso. The sweat sensor patch 220 may be easily affixed to the human body 232 using medical tape or medical adhesive. The sweat sensor patch 220 may be easily removed from the human body 232 by peeling off the sweat sensor patch 220. New sweat sensor patches 220 may be attached to the human body 232 on a periodic basis. For example, a human subject may replace the sweat sensor patch daily, weekly, or monthly. New sweat sensor patches may integrate with other existing components of a self-powered sweat sensor system. For example, a new patch may connect to existing circuitry that may connect to a power system that provides power to the sweat sensor system.

### System Integration with FTENG

The FTENG-powered wearable sweat sensor system may include various components. It may include an interdigital stator. It may also include a power-management integrated circuit (PMIC). It may also include a low dropout (LDO) voltage regulator, one or more, for example two, low-power instrumentation amplifiers, and a Bluetooth low-energy (BLE) programmed system on a chip (PSoC) module. All of these components may be seamlessly integrated onto a polyimide based flexible printed circuit board (FPCB). The system may further include a grating patterned FTENG slider and a microfluidic sensor patch.

Design of the FTENG and electronic circuitry on a single printed circuit board may allow for seamlessly interchanging the sensor patch and/or integrating other types of sensors that may be suitable for similar self-powering mechanisms. The integration of parts of or the entire system on a FPCB may allow for easier application onto the human body without comprising the effectiveness of the system. It also may allow the sweat sensor patch to be fabricated as a disposable device to be replaced frequently, while the other components, which may be more cost effective to manufacture as permanent devices, are not replaced frequently.

Because continuous monitoring has high-power needs, efficiency is relevant to an effective design. A PMIC may be included in the system to manage power generated by the FTENG so that it more efficiently powers the device while minimizing energy waste. The PMIC can store energy generated by the FTENG in one or more, for example two, parallel capacitors. Then, stored power can be released when needed using a switch control logic system. Capacitors can be disconnected and reconnected on an alternating basis when fully charged.

The sweat sensor patch system can also conserve energy by reducing power needs. Continuous monitoring requires greater energy and even more energy is required when data is transmitted wirelessly on a continuous basis, as disclosed herein. Therefore, a system may include a Bluetooth low-energy programmed system on a chip (BLE PSoC) module to maintain data transmission via Bluetooth without incurring steep energy costs.

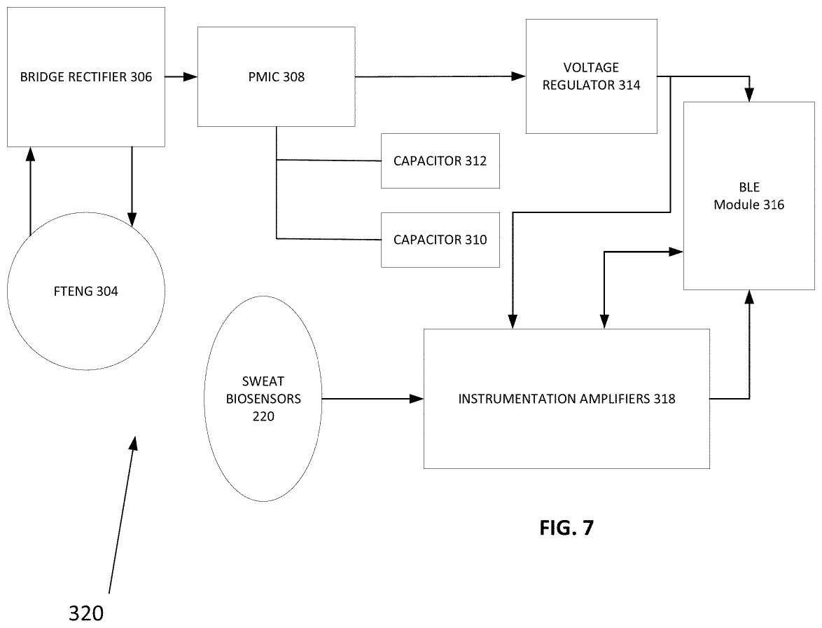

FIG. 7 shows an example of the electronic circuitry for the sweat sensor system. The FTENG 304 may power the system. The FTENG may include an interdigital stator. It may also include a grating slider. When a person wearing the sweat sensor system moves, the movement may cause the slider to slide across the stator. This may generate a charge, converting the mechanical energy of the movement into electric energy. Power from the FTENG may travel through a bridge rectifier 306. The bridge rectifier 306 may assist in converting high voltage AC signals generated by the FTENG into a DC signal. The signal may then travel to the PMIC 308. The PMIC 308 may manage energy generated by the FTENG to minimize power waste. The PMIC 308 may accomplish this more efficient power management by storing the FTENG generated energy in two capacitors 310, 312 in parallel. Resistors may be programmed such that stored power is released when certain thresholds are achieved. When the voltage of the capacitors 310, 312 storing the energy reach a threshold voltage, the capacitors 310, 312 may supply energy until their voltages reach a lower threshold. Then, the PMIC 308 may disconnect the capacitors 310, 312 until they are charged back to the upper threshold. The energy passing from the capacitors 310, 312 may then be regulated by a voltage regulator 314, which may provide the rest of the circuitry, for example the BLE PSoC Module 316 and instrumentation amplifiers 318, with a stable voltage.

### System Integration with PV Panel

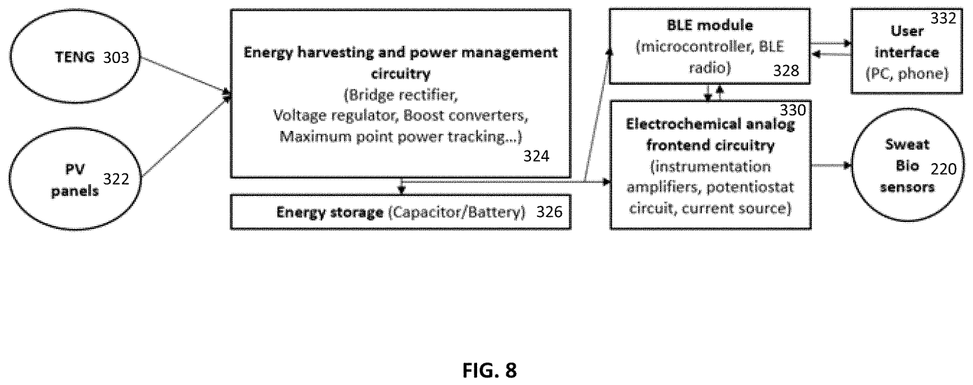

FIG. 8 shows an example of a system including both PV panels 322 and a TENG 303. A system may include, as power sources, both a TENG 303 device and photovoltaic (PV) panels 322. The PV panels 322 may be used simultaneously or alternatively to power the system. For example, a TENG 303 device may supply power while a human patient is exercising. A PV panel 322 may continue to supply power while a human patient is in a sedentary state. To manage the power supply, the system may also include several components. For example, the system may include an energy harvesting PMIC 324. The PMIC 324 may include several components, such as a bridge rectifier, a voltage regulator, boost converters, a maximum point power tracking element, and other appropriate elements to manage the power output. The system may also include energy storage components 326. For example, the system may include capacitors and/or batteries. The system may also include a BLE module 328. The BLE module 328 may include additional components, such as a microcontroller and a BLE radio. The system may also include an electrochemical AFE circuitry 330. The AFE circuitry 330 may include elements, such as instrumentation amplifiers, a potentiostat circuit, a current source, and other appropriate elements. The system may also include a user interface 332. For example, the user interface may be a mobile device or a PC. The system may also include sweat biosensors on a sweat sensor patch 220 or a smart patch, consistent with embodiments described above.

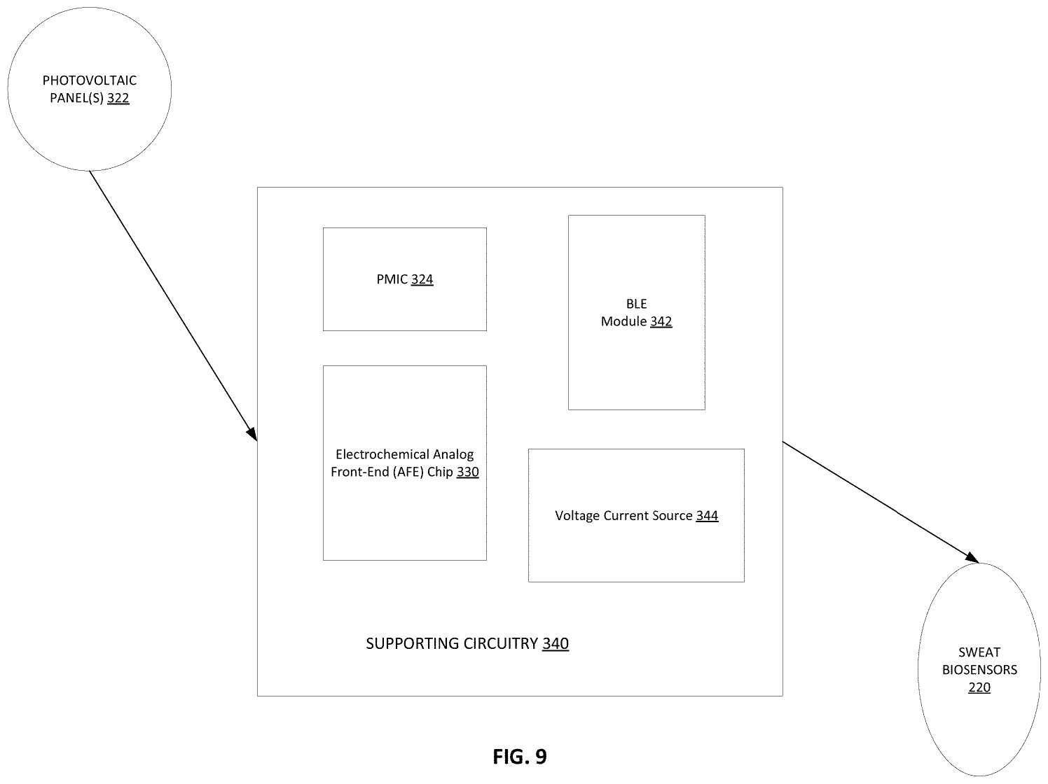

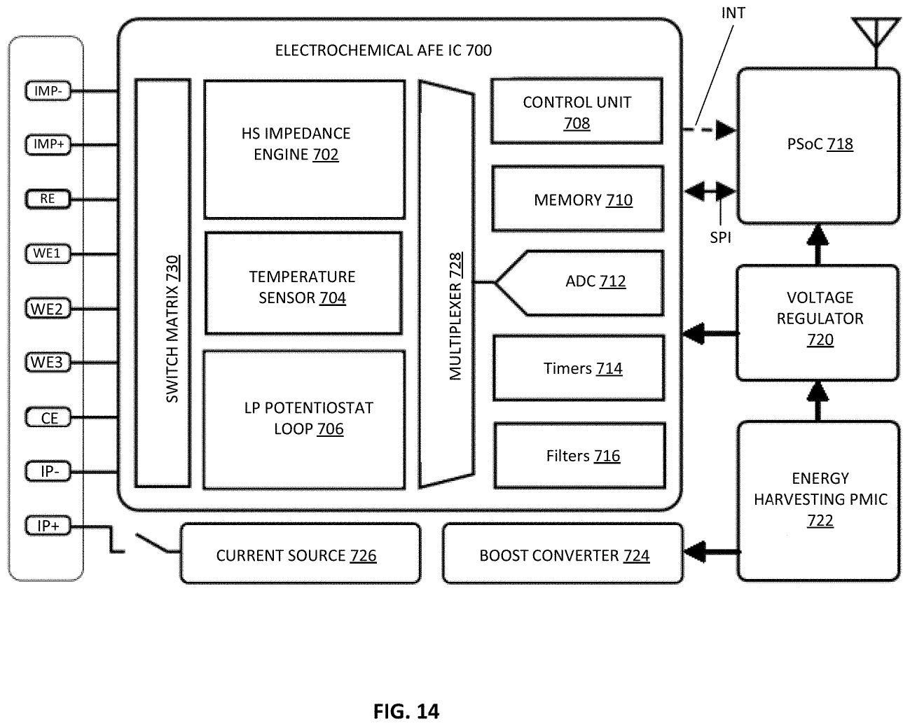

FIG. 9 shows an example of supporting circuitry 340 for the self-powered wireless biosensor. The photovoltaic panels 322 may power the system and be electrically coupled to the supporting circuitry. When a person wearing the self-powered wireless biosensor is exposed to a light source, the light energy may be converted into electric energy by the photovoltaic panels 322. Power from the photovoltaic panels 322 may travel to supporting circuitry 340 to boost, convert, and/or manage the generated power. The supporting circuitry 340 may be fabricated and integrated using PCB technology. The supporting circuitry 340 may include an energy harvesting PMIC 324. The PMIC 324 may boost, convert, and manage the power output from the photovoltaic panels 322. The supporting circuitry 340 may also include a compact PSoC BLE module 342. The BLE module 342 may integrate an MCU and a BLE radio. The supporting circuitry 340 may also include a high compliance voltage current source 344 for iontophoresis. The supporting circuitry 340 may also include an electrochemical AFE chip 330. The AFE chip 330 may integrate various configurable blocks necessary for electrochemical detection. An example AFE chip 330 is shown in FIG. 14 and is described in more detail below. The supporting circuitry 340 may supply power generated by the PV panels 322 to the sweat biosensors on a sweat sensor patch 220. This configuration may supply power for a period of over four hours. The supporting circuitry 340 may further be communicatively coupled to the wearable sensor patch, which in some embodiments, may be a sweat biosensor on a sweat sensor patch 220.

A PV panel may output a low voltage DC signal. Therefore, a PV panel may need to be boost converted to charge a capacitor. With exposure to light, a PV panel may output several milliwatts of power. This power output may be used to charge capacitors. The power output may be sufficient to continuously power a connected biosensor and supporting electronics without the need to completely discharge the capacitors to a lower threshold voltage. The power output may also support several electrochemical measurement techniques, including, for example, potentiometry, amperometry, voltammetry, impedance spectroscopy, and iontophoresis. An integrated PV panel biosensor system may achieve sufficient power output to perform, for example, iontophoresis to stimulate sweat in a sedentary human patient, perform simultaneous potentiometry and amperometry to acquire multiplexed sweat glucose, pH sodium data, and other relevant biometric data from the sweat sample, and perform impedance analysis to measure the sweat rate.

### Self-Powered Wearable Biosensor System on Human Body

The FTENG system can be attached directly to human skin. This configuration may allow for efficient powering of skin-interfacing wearables. Waterproof medical tape may be used to secure the device to human skin. The device may be secured to the human torso or another suitable place on the human body.

With respect to the FTENG system, certain types of exercise and/or movement of the human body may produce a sliding motion between the torso and the inner arm. These exercises may include, for example, running, jogging, rowing, training on an elliptical or other cardiovascular exercise type equipment, and other types of exercise. This type of movement may power the FTENG. The stator of the FTENG may be attached to the human torso. The slider of the FTENG may be attached to the inner arm such that when the human body moves, the slider slides against the stator. This sliding motion may transform the mechanical energy of the body movement into electrical energy as charge accumulates.

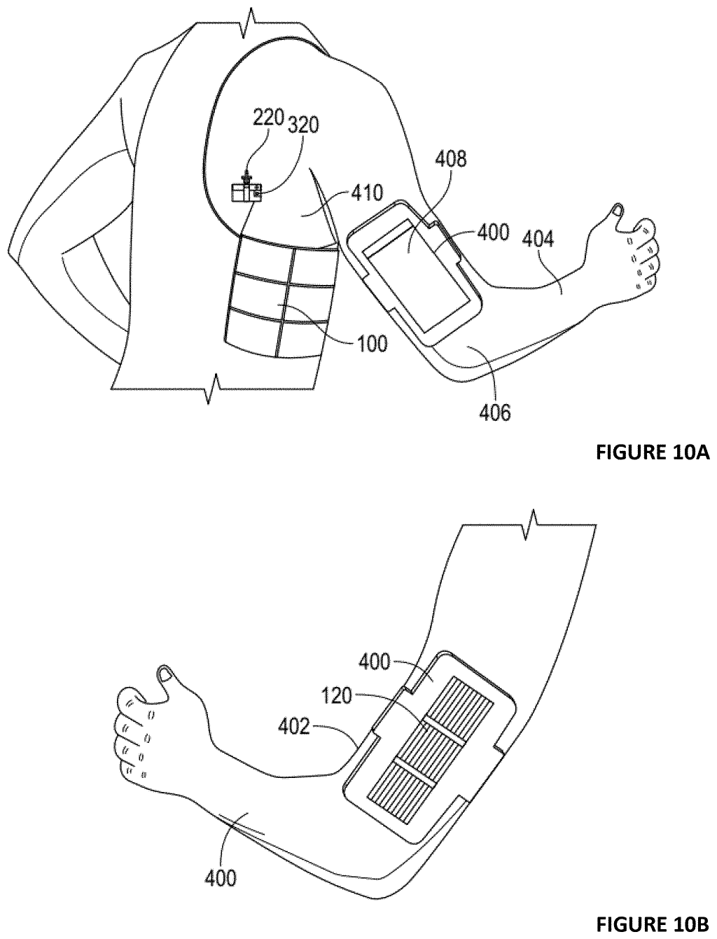

FIGS. 10A and 10B show examples of how the FTENG-powered wireless biosensor system may be worn on the body. FIG. 10A shows a 6-panel stator 100 attached to a human torso 410. The stator 100 may be connected to circuitry for example 320 or 340, which may include power management modules and may be connected to the biosensor skin patch of the sweat sensor patch 220. A human subject may also wear an arm band 400 on an arm 404. On the outer side 406 of the arm, the arm band 400 may contain a user interface 408. The user interface 408 may be part of a mobile device. FIG. 10B shows an example of the inner side 402 of the arm 404. The arm band 408 on the inner side 402 of the arm 404 may contain slider 120 panels. When the human subject moves and/or exercises in a particular way, the slider 120 panels on the inner side 402 of the arm 404 may slide against the stator 100 panels on the torso and power the system.

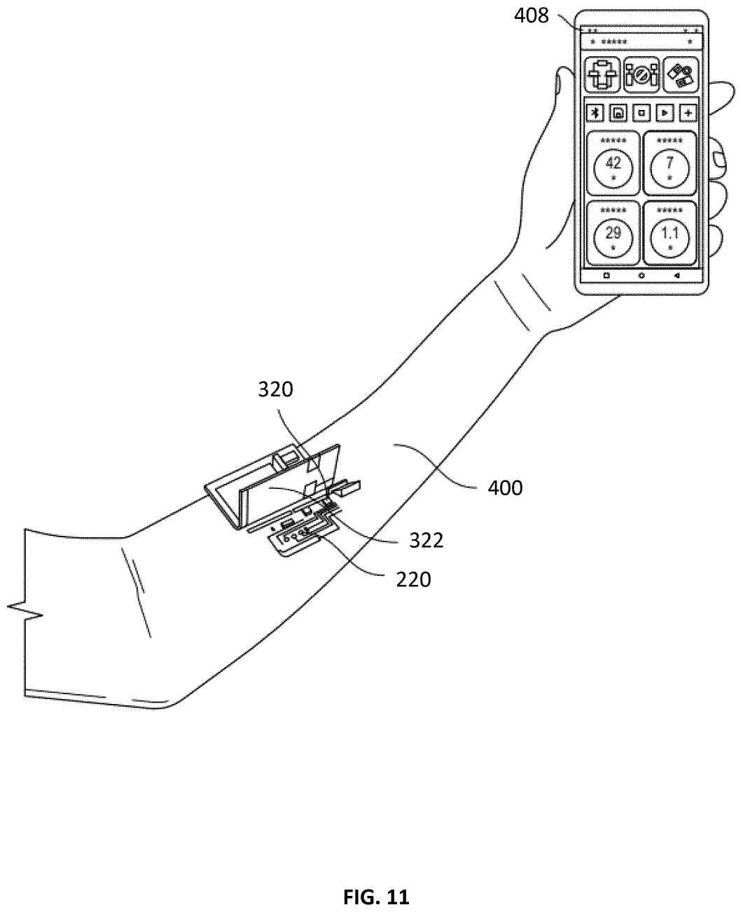

FIG. 11 shows an example of how the PV panel-powered wireless biosensor system may be worn on the body. FIG. 11 shows a photovoltaic panel 322 attached to a human arm 400. The photovoltaic panel 322 may be connected to supporting circuitry, which may be connected to the biosensor skin patch of the sweat sensor patch 220. A human subject may also operate a mobile device which may contain a user interface 408 for analyzing and viewing health information collected from the wearable biosensor system.

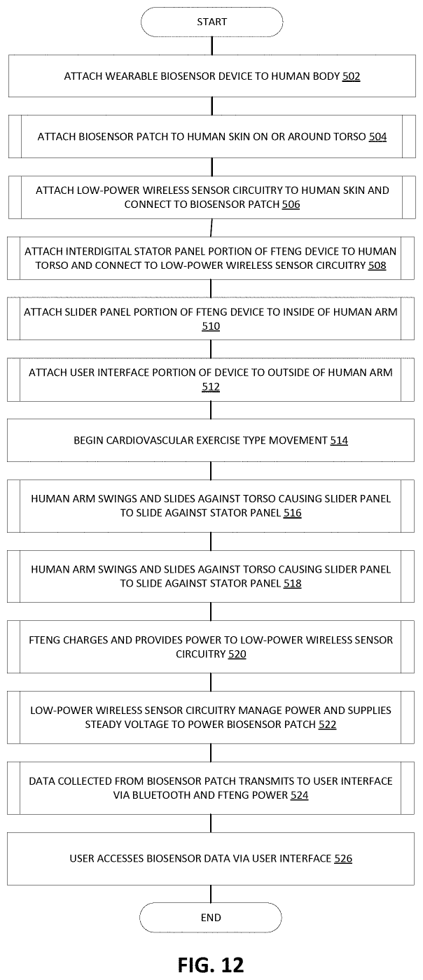

FIG. 12 is a flow diagram showing an example of a method of powering an FTENG-powered wearable biosensor system via human motion. The method may include first attaching a wearable biosensor to the human body 502. This may involve attaching a biosensor patch, such as a microfluidic sweat sensor patch, to human skin on or near the torso area of the a human body 504. This may further involve attaching a low-power wireless sensor circuitry to the human skin and connecting the low-power wireless sensor circuitry to the sensor patch 506. This may further involve attaching an interdigital stator panel portion of an FTENG powering device to the human skin and connecting the stator to the low-power wireless sensor circuitry 508. This may also involve attaching a slider panel portion of an FTENG device to the inside of a human arm 510. This may also involve attaching a user interface portion of the system to the outside of a human arm 512.

A method of powering a self-powered wearable biosensor system may then involve beginning a cardiovascular exercise type of movement, such as running 514. During movement, the human arm may naturally swing and the inside portion of the human arm may slide against a torso area on the human body 516. This motion may cause the slider panel to slide across the stator panel 518. The sliding may charge the FTENG and provide power to the low-power wireless sensor circuitry 520. The low-power wireless sensor circuitry may manage the supplied power for efficient powering of the overall system and may supply a steady voltage 522. This steady voltage may power the biosensor patch and may enable transmission of data collected form the biosensor patch to a user interface. This transmission may be accomplished via Bluetooth or another wireless method 524. A user may then access biosensor data via the user interface 526.

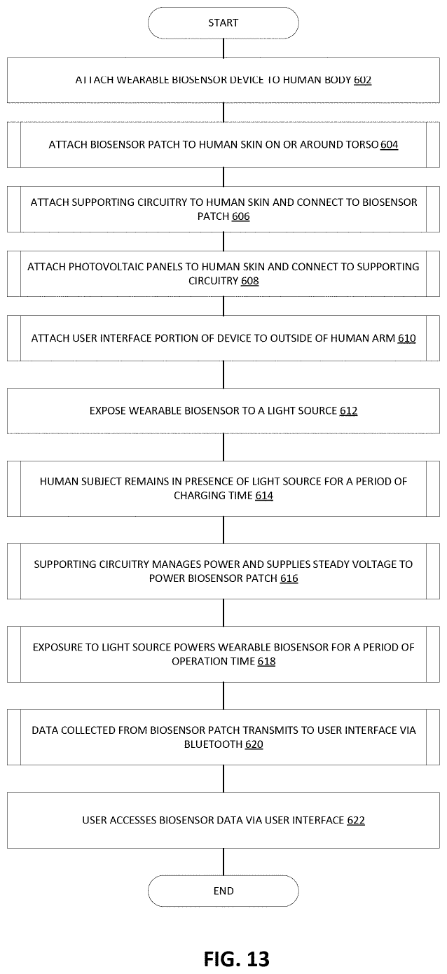

FIG. 13 is a flow diagram showing an example of a method of powering a PV panel-powered wearable biosensor system via light energy. The method may include first attaching a wearable biosensor to the human body 602. This may involve attaching a biosensor patch, such as a microfluidic sweat sensor patch, to human skin on or near the torso area of the a human body 604. A sweat sensor patch may be applied to another area of the human body. The method may further involve attaching a supporting circuitry to the human skin and connecting the supporting circuitry to the sensor patch 606. This may further involve attaching an a photovoltaic panel powering device to the human skin and connecting the photovoltaic panel array to the supporting sensor circuitry 608. This may also involve attaching a user interface portion of the system to the outside of a human arm 610. This may further involve exposing the wearable biosensor to a light source 612. The human subject may remain in the presence of the light source for a period of charging time 614. From the energy received from the power source, the supporting circuitry may manage power and supply steady voltage to power the patch 616. The patch may then be powered from this stored power for a period of operation time 618. The data collected during the operation time can then be transmitted from the biosensor patch to a user interface via Bluetooth 620, where the user may access the data via the user interface 622.

FIG. 14 shows an example of an electrochemical analog front-end (AFE) chip 700. In an embodiment, the AFE chip 700 may be connected to the PMIC 722, voltage regulator 720, and PSoC 718 to integrate blocks necessary for electrochemical detection. The PMIC 722 may also be connected to a boost converter 724. The AFE chip 700 may contain configurable amplifiers for potentiometric, amperometric, voltametric, and impedance measurements at multiple modes of measurement ranges and resolutions. For example, the AFE may include an HS impedance engine 702, a temperature sensor 704, and an LP potentiostat loop 706. Additionally, a current source 726 may support the amplifiers. The AFE chip 700 may also include a switch matrix 730 and a multiplexer 728 to flexibly connect sensors and analog signals to the appropriate channels. For example, a lower power current measurement channel may be connected to an external current source to monitor iontophoresis current. The AFE chip 700 may also include other elements, such as a sequencer, a control unit 708, a memory block 710, a converter 712, a waveform generator, timers 714, filters 716, and a DFT hardware accelerator. The DFT hardware accelerator may enable independent operation of complex electrochemical procedures and may minimize the workload of the microcontroller and the overall power consumption. The PSoC Bluetooth module 718 may act as a data bridge between the electrochemical AFE 700 and a host software, for instance a mobile phone or PC, such that it encodes and writes measurement instructions to the AFE and then decodes and transmits the AFE's measurements to the host software via BLE.

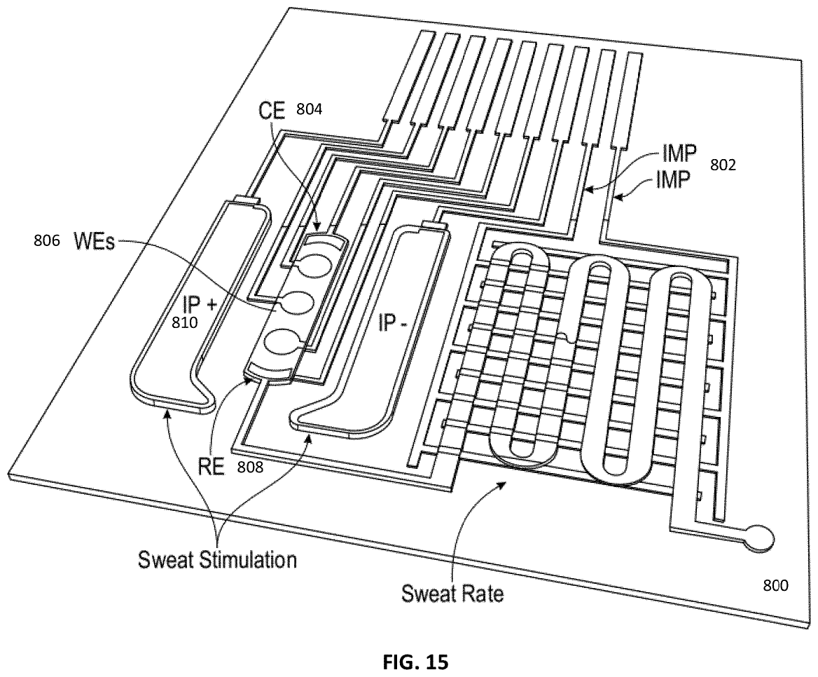

FIG. 15 shows an example of a disposable microfluidic sweat patch 800 for sweat induction and sampling. The patch may include impedance (IMP) 802, a counter electrode (CE) 804, a working electrode (WE) 806, a reference electrode (RE) 808, and an iontophoresis module (IP) 810. The patch 800 may be assembled using off-the-shelf electronic components. In some embodiments, the patch 800 may be affixed to a skin area with adhesive. In some embodiments, the patch may be powered by and/or interface with a self-power and/or battery-free power means. In some embodiments, the power means may be a perovskite solar cell in accordance with above described embodiments. In some embodiments, the patch may be fabricated and/or printed using an inkjet printer. In some embodiments, the patch may include two gel-loaded iontophoretic electrodes, three electrochemical sweat biosensors, and one sweat rate sensor embedded in the microfluidics of the patch. Other numbers of sensors may exist.

While various embodiments of the present invention have been described above, it should be understood that they have been presented by way of example only, and not of limitation. Likewise, the various diagrams may depict an example architectural or other configuration for the invention, which is done to aid in understanding the features and functionality that can be included in the invention. The invention is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present invention. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects, and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known,” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to,” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

The terms “substantially,” “approximately,” and “about” are used throughout this disclosure, including the claims, are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

What is claimed is:

1. A self-powered wearable system, comprising:
a wearable sensor patch;
supporting circuitry communicatively coupled to the wearable sensor patch;
a photovoltaic panel electrically coupled to the supporting circuitry; and
a motion power component including a stator and a slider, wherein the motion power component produces current when the slider moves across the stator.

2. The self-powered wearable system of claim 1, wherein the wearable sensor patch further comprises a microfluidic sweat sensor patch.

3. The self-powered wearable system of claim 1, wherein the supporting circuitry further comprises:
a power management integrated circuit (PMIC);
an electrochemical analog front-end (AFE) chip;
a Bluetooth low-energy programmed system on a chip (BLE) module; and
a voltage current source.

4. The self-powered wearable system of claim 1, wherein the photovoltaic panel further comprises a perovskite solar cell.

5. The self-powered wearable system of claim 1, wherein the motion power component is a freestanding triboelectric nanogenerator (FTENG).

6. The self-powered wearable system of claim 1, further comprising a user interface wherein the user interface wirelessly receives sample data collected by the wearable sensor patch.

7. The self-powered wearable system of claim 1, wherein the photovoltaic panel, motion power component, and supporting circuitry supply a stable voltage to the wearable sensor patch for a period of time.

8. The self-powered wearable system of claim 7, further comprising a battery wherein the photovoltaic panel and motion power component supply the battery with power and where the power supplied by the photovoltaic panel and motion power component is stored in the battery.

9. The self-powered wearable system of claim 1, wherein the wearable sensor patch, supporting circuitry, photovoltaic panel, and motion power component are supported on integrated platform leveraging printed circuit board (PCB) technology.

10. An autonomous sweat sampling method, comprising:
collecting power from a light source with a wearable photovoltaic panel;
converting the power collected from the light source into electrical energy with a supporting circuitry connected to the wearable photovoltaic panel;
powering a wearable microfluidic sweat sensor patch connected to the supporting circuitry, wherein the microfluidic sweat sensor patch collects human sweat samples and analyzes the collected samples to monitor and identify health factors; and
repeating the above method steps for continuous collection, analysis, and monitoring of human sweat samples over a period of time.

11. The autonomous sweat sampling method of claim 10, wherein the supporting circuitry comprises an electrochemical analog front-end (AFE) chip.

12. The autonomous sweat sampling method of claim 10, wherein the light source comprises an artificial light source.

13. The autonomous sweat sampling method of claim 10, further comprises collecting power from human movement with a wearable freestanding triboelectric nanogenerator (FTENG) and converting the power collected from the artificial light source into electrical energy with supporting circuitry connected to the wearable freestanding triboelectric nanogenerator (FTENG).

14. An autonomous biometric monitoring method comprising:
wearing a wearable biometric monitoring device, the wearable device comprising:
a photovoltaic panel;
an FTENG component;
supporting circuitry; and
a microfluidic sweat sensor patch,
wherein the photovoltaic panel, FTENG component, supporting circuitry, and microfluidic sweat patch are all supported on integrated platform leveraging printed circuit board (PCB) technology; and
exposing the wearable device to a light source for a period of charging time, wherein exposure to the light source powers the wearable biometric monitoring device for a period of operation time, and
wherein moving the microfluidic sweat sensor patch powers the wearable biometric monitoring device via collected energy for a period of operation time.

15. The method of claim 14, wherein the light source is an artificial light source.

16. The method of claim 14, wherein the FTENG component powers the wearable biometric monitoring device via collected energy for a period of operation time.

17. The method of claim 14, wherein the period of operation time is based on a period of charging time from the photovoltaic panel and the FTENG component.

18. The method of claim 14, wherein wearing the wearable device further comprises applying the photovoltaic panels to an exposed area of skin on a human arm.

19. The method of claim 14, wherein wearing the wearable device further comprises applying the photovoltaic panels to an area on a human torso.

20. The method of claim 14 further comprising accessing sample data collected by the wearable device using a user interface.
